(19) United States
(12) Patent Application Publication
(10) Pub. No.: US 2024/0294453 A1
(43) Pub. Date: Sep. 5, 2024

(54) Deuterated agonists and methods of use

(71) Applicant: Jeffrey Alan McKinney, Half Moon Bay, CA (US)

(72) Inventor: Jeffrey Alan McKinney, Half Moon Bay, CA (US)

(21) Appl. No.: 18/445,729

(22) Filed: Jan. 2, 2024

Related U.S. Application Data
(63) Continuation of application No. 16/602,652, filed on Nov. 15, 2019, now abandoned.
(60) Provisional application No. 62/810,212, filed on Feb. 25, 2019.
(60) Provisional application No. 62/797,666, filed on Jan. 28, 2019.
(60) Provisional application No. 62/796,811, filed on Jan. 25, 2019.
(60) Provisional application No. 62/917,463, filed on Dec. 7, 2018.

(51) Int. Cl. C07C 39/23; C07B 59/00

(52) U.S. Cl. CPC C07C 39/23; C07B 59/001; C07B 2200/05; C07C 2601/16

(57) ABSTRACT

The present invention is generally directed to deuterated agonists and their methods of use. It is more specifically directed to deuterated alpha-7 nicotinic receptor agonists and their methods of use. In one aspect, the present invention involves compounds of the structure 1-34 as shown in FIGS. 1-4. In another aspect, the present invention involves the treatment of a disorder with a compound of the structure 1-34 as shown in FIGS. 1-4.

This application claims the benefit of U.S. Provisional Application No. 62/917,463, filed Dec. 7, 2018, entitled “Deuterated Compounds and Methods of Use”, and claims the benefit of U.S. Provisional Application No. 62/796,811, filed Jan. 25, 2019, entitled “CB and Alpha 7NACHR Agonists”, and claims the benefit of U.S. Provisional Application No. 62/797,666, filed Jan. 28, 2019, entitled “Deuterated Agonists and Methods of Use”, and claims the benefit of U.S. Provisional Application No. 62/810,212, filed Feb. 25, 2019, entitled “Deuterated Agonists and Methods of Use”, each of which is incorporated-by-reference into this application for all purposes.

### FIELD OF THE INVENTION

The present invention is generally directed to deuterated agonists and their methods of use. It is more specifically directed to deuterated alpha-7 nicotinic receptor agonists and their methods of use.

### BACKGROUND OF THE INVENTION

There have been reports of alpha-7 nicotinic receptor agonists. For instance, the article “Phase 2 Trial of an Alpha-7 Nicotinic Receptor Agonist (TC-5619) in Negative and Cognitive Symptoms of Schizophrenia”, Walling et al. Schizophrenia Bulletin, 42(2) June 2015, allegedly reports the following: “This trial was conducted to test the effects of an alpha7 nicotinic receptor full agonist, TC-5619, on negative and cognitive symptoms in subjects with schizophrenia. In 64 sites in the United States, Russia, Ukraine, Hungary, Romania, and Serbia, 477 outpatients (18-65 years; male 62%; 55% tobacco users) with schizophrenia, treated with a new-generation antipsychotic, were randomized to 24 weeks of placebo (n=235), TC-5619, 5 mg (n=121), or TC-5619, 50 mg (n=121), administered orally once daily. The primary efficacy measure was the Scale for the Assessment of Negative Symptoms (SANS) composite score. Key secondary measures were the Cogstate Schizophrenia Battery (CSB) composite score and the University of California San Diego Performance-Based Skills Assessment-Brief Version (UPSA-B) total score. Secondary measures included: Positive and Negative Syndrome Scale in Schizophrenia (PANSS) total and subscale scores, SANS domain scores, CSB item scores, Clinical Global Impression-Global Improvement (CGI-I) score, CGI-Severity (CGI-S) score, and Subject Global Impression-Cognition (SGI-Cog) total score. SANS score showed no statistical benefit for TC-5619 vs placebo at week 24 (5 mg, 2-tailed P=0.159; 50 mg, P=0.689). Likewise, no scores of CSB, UPSA-B, PANSS, CGI-I, CGI-S, or SGI-Cog favored TC-5619 (P>0.05). Sporadic statistical benefit favoring TC-5619 in some of these outcome measures were observed in tobacco users, but these benefits did not show concordance by dose, country, gender, or other relevant measures. TC-5619 was generally well tolerated. These results do not support a benefit of TC-5619 for negative or cognitive symptoms in schizophrenia.” Abstract.

Despite the reports of alpha-7 nicotinic receptor agonists, there is still a need in the art for novel compounds and related methods.

### BRIEF SUMMARY OF THE INVENTION

In one aspect, the present invention involves compounds of the structure 1-70 and 100-179 as shown in FIGS. 1-17.

In another aspect, the present invention involves the treatment of a disorder with a compound of the structure 1-70 and 100-179 as shown in FIGS. 1-17.

### BRIEF DESCRIPTION OF THE DRAWINGS

FIG. 1 shows an alpha-7 nicotinic agonist (1) and related deuterated compounds (2-7).

FIG. 2 shows deuterated alpha-7 nicotinic agonists (8-16).

FIG. 3 shows deuterated alpha-7 nicotinic agonists (17-25).

FIG. 4 shows deuterated alpha-7 nicotinic agonists (26-34).

FIG. 5 shows an alpha-7 nicotinic agonist (35) and related deuterated compounds (36-42).

FIG. 6 shows deuterated alpha-7-nicotinic agonists (43-50).

FIG. 7 shows deuterated alpha-7-nicotinic agonists (51-60).

FIG. 8 shows deuterated alpha-7-nicotinic agonists (61-70).

FIGS. 9-14 show various synthetic routes to agonists.

FIGS. 15-17 show various synthetic intermediates for the synthesis of agonists.

### DETAILED DESCRIPTION OF THE INVENTION

“Combined Dose” in reference to administration of two or more compounds, refers to the total dose of the two or more compounds provided together. For instance, if a combined dose of a deutero cannabidiol and a deutero tetrahydrocannabinol ranges from 10 mg to 100 mg, then the provided mass of deutero cannabidiol and deutero tetrahydrocannabinol added together ranges from 10 mg to 100 mg.

“Other Suitable Index” or “Other Suitable Measure” refers to another index, rating system, or scale that measures benefit of treatment for a particular disease state (e.g., anxiety, depression).

“Non-Deutero” or “Non-Deuterium Enriched” in reference to a compound means that the compound has a naturally occurring isotopic abundance of protium, deuterium and tritium where a hydrogen atom is present. For instance, the hydrogen atoms in a non-deutero or non-deuterium enriched cannabidiol have a naturally occurring isotopic abundance (i.e., greater than 99 percent, or approximately 99.98, percent protium, and less than 1 percent, or approximately 0.02 percent, deuterium).

The compounds of the present invention include deuterium in an amount that is more than expected from isotopic abundance. This deuterium enrichment can be shown by inclusion of “D” in a chemical structure. Typically, in the case of deuterium enrichment, the amount of deuterium (e.g., where “D” is indicated), as opposed to protium or tritium, is more than 20 percent, more than 30 percent, more than 40 percent, more than 50 percent, more than 60 percent, more than 70 percent, more than 80 percent, more than 90 percent or more than 95 percent.

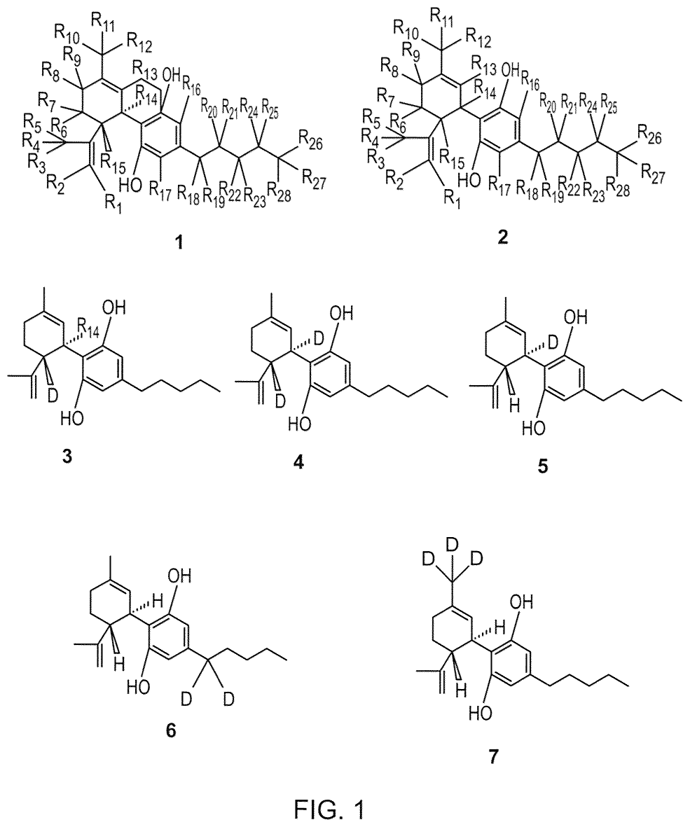

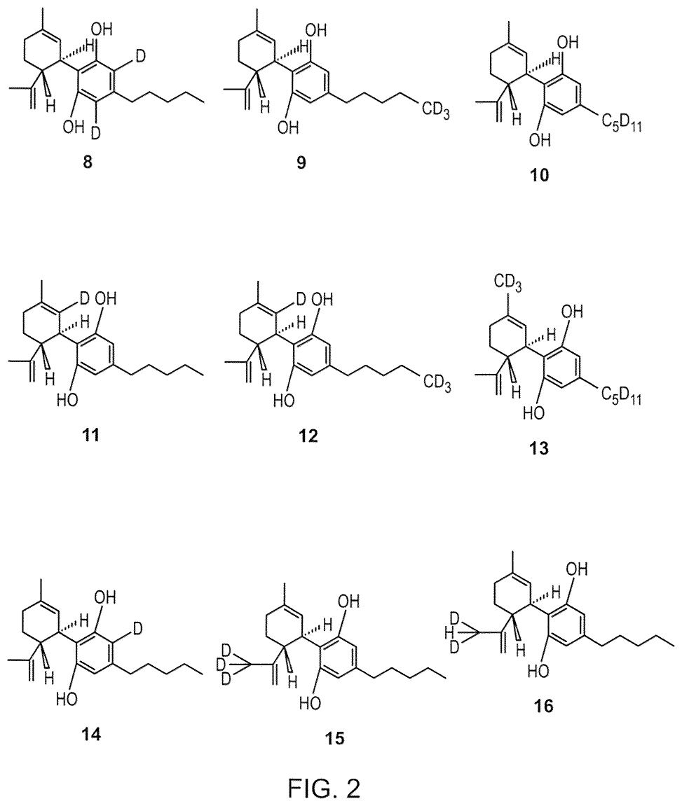

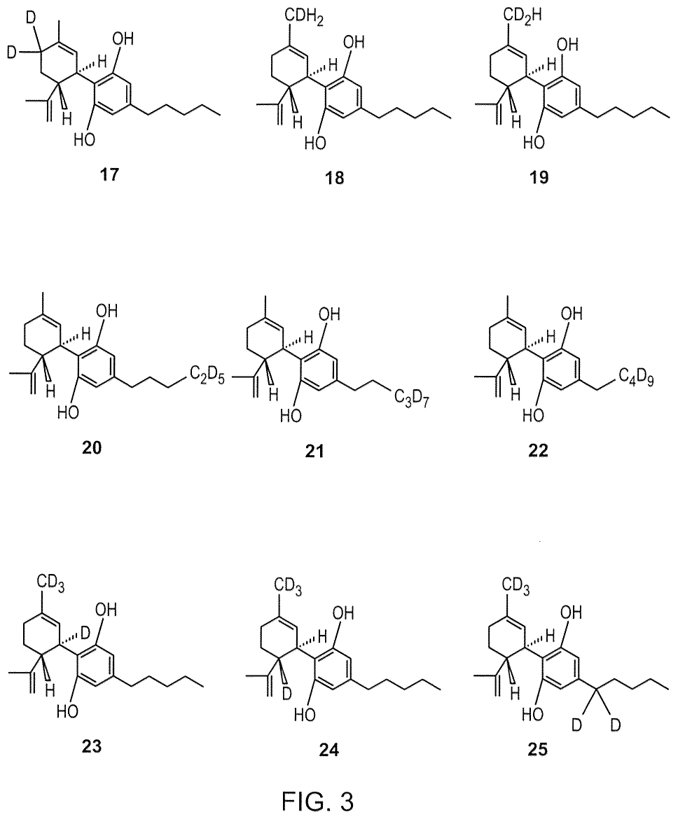

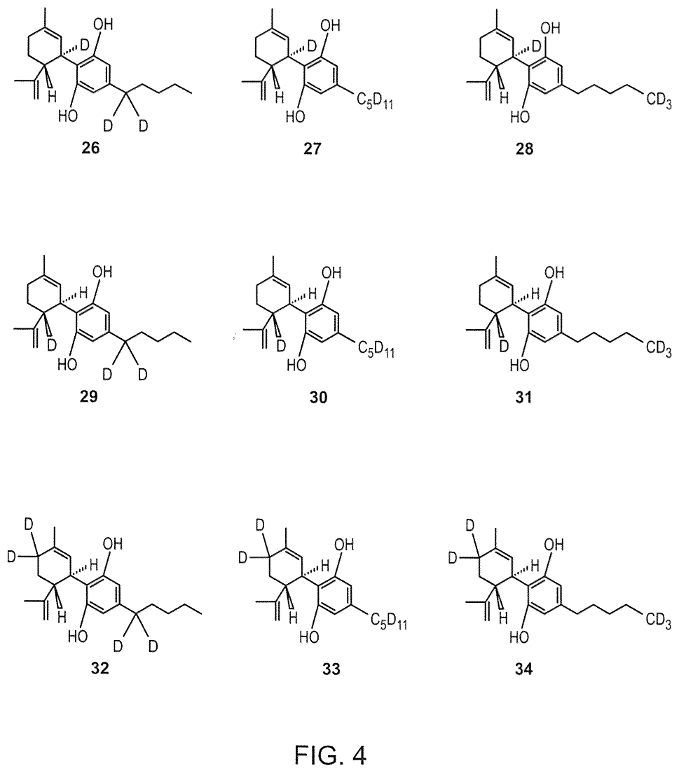

The present invention provides deuterated alpha-7 nicotinic receptor agonists. In reference to FIG. 1: Compound 1—R₁ to R₂₈ are independently “H” or “D”, where at least one of the substituents is “D”. Compound 2—R₁ to R₂₈ are independently “H” or “D”, where at least one of the substituents is “D”. Compound 3—R₁₄ is “H” or “D”.

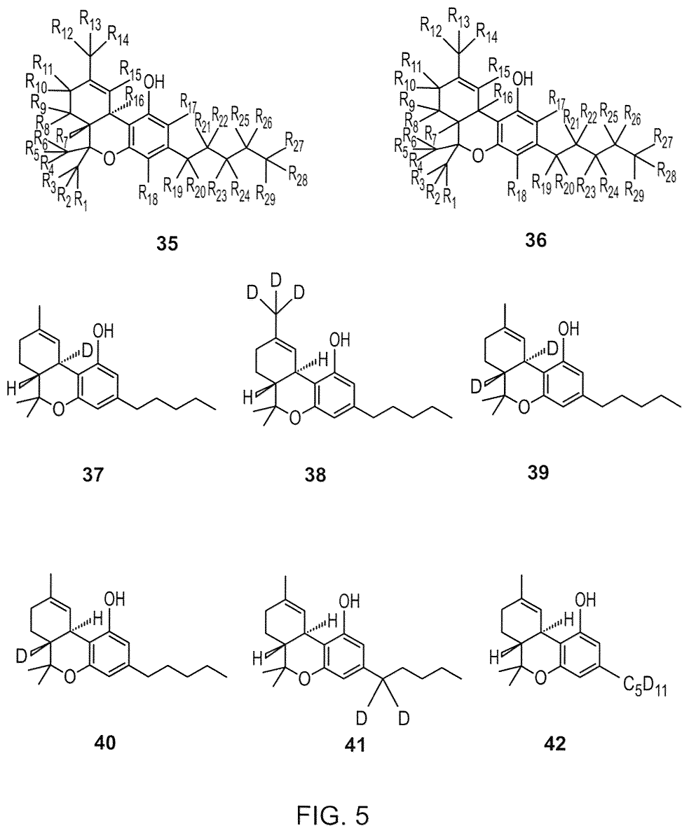

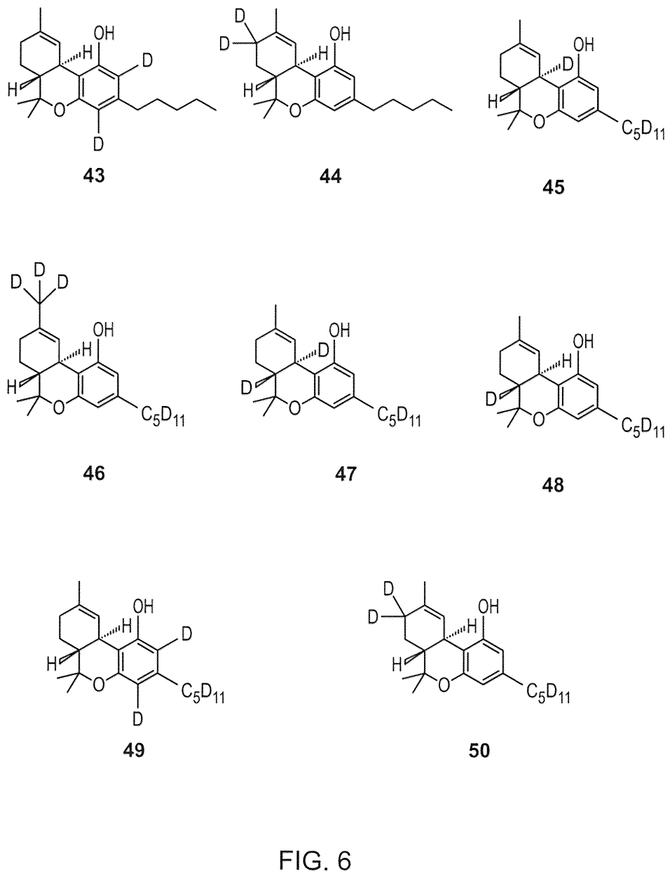

In reference to FIG. 5: Compound 35—R₁ to R₂₉ are independently “H” or “D”, where at least one of the substituents is “D”. Compound 36—R₁ to R₂₉ are independently “H” or “D”, where at least one of the substituents is “D”.

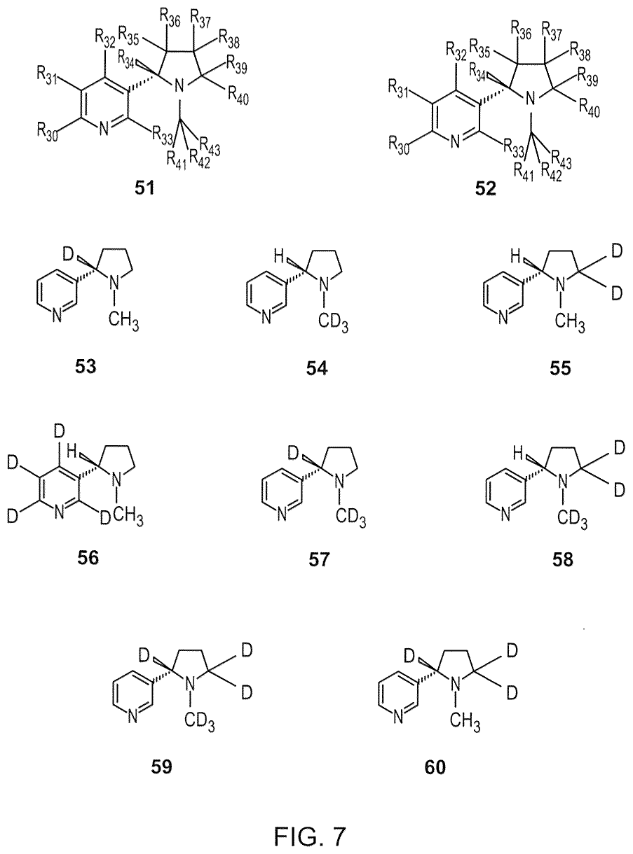

In reference to FIG. 7: Compound 51—R₃₀-R₄₃ are independently “H” or “D”, where at least one of the substituents is “D”. Compound 52—R₃₀-R₄₃ are independently “H” or “D”, where at least one of the substituents is “D”.

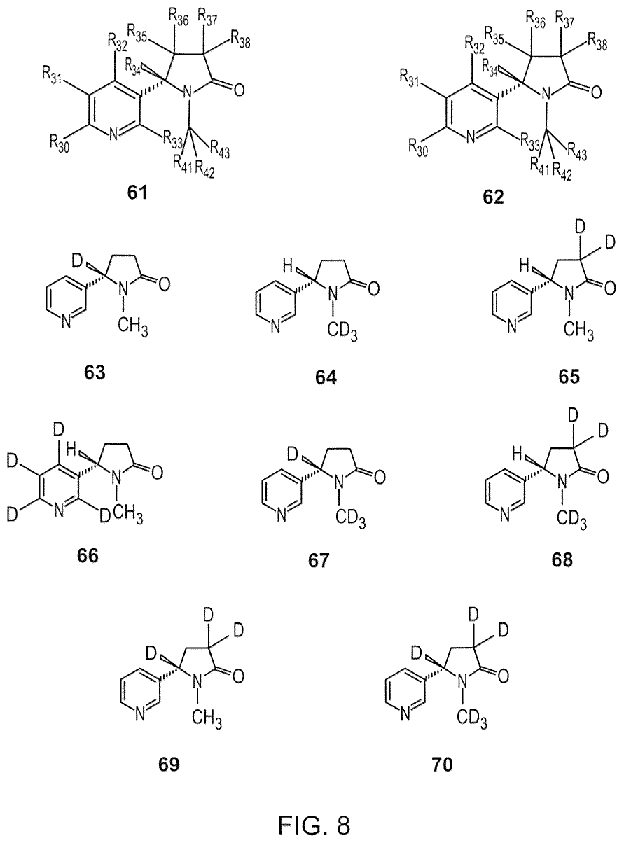

In reference to FIG. 8: Compound 61—R₃₀-R₃₈ and R₄₁-R₄₃ are independently “H” or “D”, where at least one of the substituents is “D”. Compound 52—R₃₀-R₃₈ and R₄₁-R₄₃ are independently “H” or “D”, where at least one of the substituents is “D”.

Compounds of the present invention can be used to treat a variety of disorders, including, without limitation, the following: pain; hyperalgesia; allodynia; inflammatory hyperalgesia; neuropathic hyperalgesia; acute nociception; osteoporosis; multiple sclerosis-related spacicity; autoimmune disorders; allergic reactions; CNS inflammation; atherosclerosis; age-related macular degeneration; cough; leukemia; lymphoma; CNS tumors; prostate cancer; Alzheimer's disease; dementia; amyotrophic lateral sclerosis; Parkinson's disease; osteoarthritis; gastrointestinal motility.

For instance, where the compound is a deuterated cannabidiol (i.e., cannabidiol where one or more hydrogen atoms has been replaced by a deuterium atom, e.g., compounds 1-34), it can be used for at least the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; and Dystonia. For such use, the dose, as compared to non-deutero cannabidiol, is typically at least 5 percent, 10 percent, 15 percent, 20 percent, or 25 percent less while providing the same or similar therapeutic benefit.

For instance: when used to treat anxiety, a deuterated cannabidiol tincture of a concentration of 4.45 mg/ml or less can be provided to change symptoms as measured by the Beck Anxiety Inventory; when used as an adjunct for bipolar depression, a deuterated cannabidiol can be provided as an oral formulation at a dose of less than 275, 250, 225 or 200 mg/day to observe a change in the Montgomery-Asberg Depression Rating Scale; when used to treat Sturge-Weber syndrome, a deuterated cannabidiol in an oral formulation can be given at a dose of 1 mg/kg/day to 20 mg/kg/day to observe a reduction in seizure frequency; when used to treat Treatment-Resistant Childhood Absence Seizures, a deuterated cannabidiol in oral formulation can be given at a dose of 15 mg/kg/day to 35 mg/kg/day to observe a reduction in Absence Seizure Counts assessed by Video-electroencephalogram; when used to treat Graft Versus Host Disease, a deuterated cannabidiol in oral formulation can be given at a dose less than 7.5 mg twice daily to observe a reduction in the percentage of patients with acute GVHD; when used to treat Prader-Willi Syndrome, a deuterated cannabidiol in oral formulation can be given at a dose less than 15 mg/kg/day to observe a reduction in months to walking and/or months to talking; when used to treat Inflammatory Bowel Disease (e.g., Crohn's Disease, Ulcerative Colitis, a deuterated cannabidiol in oral formulation can be given at a dose less than 4 mg twice daily to observe a reduction in Crohn's Disease Activity Index; when used to treat Refractory Infantile Spasms, a deuterated cannabidiol in oral formulation can be given at a dose less than 7.5 mg/kg to 35 mg/kg twice a day to observe a reduction of spasms and/or hypsarrythmia as confirmed by video-electroencephalogram; when used to treat Dravet Syndrome, a deuterated cannabidiol in oral formulation can be given at a dose less than 7.5 mg/kg/day to observe a reduction in the frequency of tonic-clonic, clonic and focal seizures with motor components; when used to treat Lennox-Gastaut Syndrome, a deuterated cannabidiol in oral formulation can be given at a dose less than 7.5 mg/kg/day to observe a reduction in frequency of motor seizures involving the trunk or extremities; when used to treat chronic pain, a deuterated cannabidiol in oral formulation can be given at a dose less than 150 mg twice a day to observe pain relief; when used to treat schizophrenia, a deuterated cannabidiol in oral formulation can be given at a dose less than 150 mg twice a day to observe a beneficial change in the Positive and Negative Syndrome Scale; when used to treat Multiple Sclerosis, a deuterated cannabidiol in oral formulation can be given at a dose less than 1.5 mg to 100 mg daily to observe a reduction in VAS scores; when used to treat Osteoarthritis, a deuterated cannabidiol in oral formulation can be given at a dose less than 150 mg twice a day to observe a reduction in transient joint inflammation and/or MIA-induced joint pain; when used to treat Dystonia, a deuterated cannabidiol in oral formulation can be given at a dose less than 75 mg to 500 mg/day to observe a reduction in dystonic symptoms. Typically, when the deuterated cannabidiol is administered through an inhalation route (compound in vapor or aerosol), the doses noted above can be reduced by at least 10, 20, 30, 40, 50, 60, 70 or 80 percent.

For instance, where the compound is a deuterated tetrahydrocannabinol (i.e., tetrahydrocannabinol where one or more hydrogen atoms has been replaced by a deuterium atom, e.g., compounds 35-50), it can be used for at least the following disorders: anxiety; glaucoma; insomnia; low appetite; muscle spasticity; nausea and pain. For such use, the dose, as compared to non-deutero tetrahydrocannabinol, is typically at least 5 percent, 10 percent, 15 percent, 20 percent, or 25 percent less while providing the same or similar therapeutic benefit.

Where the deuterated tetrahydrocannabinol is taken orally, the dose taken is typically between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg. Where the deuterated tetrahydrocannabinol is taken through inhalation (e.g., vaping/smoking), the dose is typically between 1 mg and 100 mg. In certain cases, the dose is between about 1 mg and about 75 mg, about 1 mg and about 50 mg, about 1 mg and about 25 mg, about 1 mg and about 10 mg, about 1 mg and about 5 mg.

In certain cases, a composition comprising at least one deuterated cannabidiol and at least one deuterated tetrahydrocannabinol is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one deuterated tetrahydrocannabinol is synergistic.

In certain cases, a composition comprising at least one deuterated cannabidiol and at least one non-deuterium enriched tetrahydrocannabinol is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one non-deuterium enriched tetrahydrocannabinol is synergistic.

Where the compound is a deuterated nicotine (i.e., nicotine where one or more hydrogen atoms has been replaced by a deuterium atom, e.g., compounds 51-60), it can be used for at least the following disorders: depression; PTSD; schizophrenia; Alzheimer's disease; Parkinson's disease; pain; weight control; and, ulcerative colitis. For such use, the dose, as compared to non-deutero nicotine, is typically at least 5 percent, 10 percent, 15 percent, 20 percent, or 25 percent less while providing the same or similar therapeutic benefit.

Where the deuterated nicotine is taken via buccal or sublingual administration, the dose taken is typically between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg. Where the deuterated nicotine is taken via patch, the dose taken is typically between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg. Where the deuterated nicotine is taken through inhalation (e.g., vaping/smoking), the dose taken is between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg.

Where the compound is a deuterated cotinine (i.e., cotinine where one or more hydrogen atoms has been replaced by a deuterium atom, e.g., compounds 61-70), it can be used for at least the following disorders: depression; PTSD; schizophrenia; Alzheimer's disease; Parkinson's disease; pain; weight control; and, ulcerative colitis. For such use, the dose, as compared to non-deutero cotinine, is typically at least 5 percent, 10 percent, 15 percent, 20 percent, or 25 percent less while providing the same or similar therapeutic benefit.

Where the deuterated cotinine is taken via buccal or sublingual administration, the dose taken is typically between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1′ mg and about 10 mg. Where the deuterated cotinine is taken via patch, the dose taken is typically between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg. Where the deuterated cotinine is taken through inhalation (e.g., vaping/smoking), the dose taken is between about 1 mg and about 250 mg. In certain cases, the dose is between about 1 mg and about 200 mg, about 1 mg and about 150 mg, about 1 mg and about 100 mg, about 1 mg and about 75 mg, about 1 mg and about 50 mg, about 1 mg and about 25 mg, about 1 mg and about 10 mg.

In certain cases, a composition comprising at least one deuterated nicotine and at least one deuterated cotinine is administered/taken to/by a person who has one of the following disorders: depression; PTSD; schizophrenia; Alzheimer's disease; Parkinson's disease; pain; weight control; and, ulcerative colitis. In these cases, the activity of the at least one deuterated nicotine and at least one deuterated cotinine is synergistic.

In certain cases, a composition comprising at least one deuterated nicotine and at least one non-deuterium enriched cotinine is administered/taken to/by a person who who has one of the following disorders: depression; PTSD; schizophrenia; Alzheimer's disease; Parkinson's disease; pain; weight control; and, ulcerative colitis. In these cases, the activity of the at least one deuterated nicotine and at least one non-deuterium enriched cotinine is synergistic.

In certain cases, a composition comprising at least one deuterated cannabidiol and at least one deuterated nicotine is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one deuterated nicotine is synergistic.

In certain cases, a composition comprising at least one deuterated cannabidiol and at least one non-deuterium enriched nicotine is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one non-deuterium enriched nicotine is synergistic.

In certain cases, a composition comprising at least one deuterated cannabidiol and at least one deuterated cotinine is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one deuterated cotinine is synergistic.

In certain cases, a composition comprising at least one deuterated cannabidiol and at least one non-deuterium enriched cotinine is administered/taken to/by a person who has one of the following disorders: anxiety; bipolar depression; Sturge-Weber syndrome; Treatment-Resistant Childhood Absence Seizures; Graft Versus Host Disease; Prader-Willi Syndrome; Drug-Resistant Epilepsy; Crohn's Disease; Ulcerative Colitis; Inflammatory Bowel Disease; Refractory Infantile Spasms; Dravet Syndrome; Lennox-Gastaut Syndrome; Chronic Pain; Schizophrenia; Atherosclerosis; Multiple Sclerosis; Osteoarthritis; Glaucoma; Insomnia; Nausea; and Dystonia. In these cases, the activity of the at least one deuterated cannabidiol and at least one non-deuterium enriched cotinine is synergistic.

Compounds of the present invention can be synthesized using methods known to one of ordinary skill in the art. Nonlimiting examples of such methods can be found in the following articles: Miyashita, M.; Sasaki, M.; Hattori, I.; Sakai, M.; Tanino, K. Science 2004, 305, 495; Foster, A. B. Trends Pharmacol. Sci. 1984, 5, 524; Kushner, D. J.; Baker, A.; Dunstall, T. G. Can. J. Physiol. Pharmacol. 1999, 77, 79; Harbeson, S. L.; Tung, R. D. Annu. Rep. Med. Chem. 2011, 46, 403; Meanwell, N. A. J. Med. Chem. 2011, 54, 2529; Phillips, D. H.; Potter, G. A.; Horton, M. N.; Hewer, A.; Crofton-Sleigh, C.; Jarman, M.; Venitt, S. Carcinogenesis 1994, 15, 1487; Jarman, M.; Poon, G. K.; Rowlands, M. G.; Grimshaw, R. M.; Horton, M. N.; Potter, G. A.; McCague, R. Carcinogenesis 1995, 16, 683; Mutlib, A. E.; Gerson, R. J.; Meunier, P. C.; Haley, P. J.; Chen, H.; Gan, L. S.; Davies, M. H.; Gemzik, B.; Christ, D. D. et al. Toxicol. Appl. Pharmacol. 2000, 169, 102; Mutlib, A. E.; Gerson, R. J.; Meunier, P. C.; Haley, P. J.; Chen, H.; Gan, L. S.; Davies, M. H.; Gemzik, B.; Christ, D. D. et al. Toxicol. Appl. Pharmacol. 2000, 169, 102; Maltais, F.; Jung, Y. C.; Chen, M .; Tanoury, J.; Perni, R. B.; Mani, N.; Laitinen, L.; Huang, H.; Liao, S.; Gao, H. et al. J. Med. Chem. 2009, 52, 799; Katsnelson, A. Nature Med. 2013, 19, 656; Atzrodt, J.; Derdau, V.; Fey, T.; Zimmermann, J. Angew. Chem. Int. Ed. 2007, 46, 7744; Atzrodt, J.; Derdau, V. J. Label. Compd. Radiopharm. 2010, 53, 67; Kluger, R. J. Org. Chem. 1964, 29, 2045; Paulsen, P. J.; Cooke, W. D. Anal. Chem. 1963, 35, 1560; Yung, C. M.; Skaddan, M. B.; Bergman, R. G. J. Am. Chem. Soc. 2004, 126, 13033; Skaddan, M. B.; Yung, C. M.; Bergman, R. G. Org. Lett. 2004, 6, 11; Heys, R. J. Chem. Soc., Chem. Commun. 1992, 68; Shu, A. Y. L.; Chen, W.; Heys, J. R. J. Organomet. Chem. 1996, 524, 87; Ma, S.; Villa, G.; Thuy-Boun, P. S.; Homs, A .; Yu, J.-Q. Angew. Chem. Int. Ed. 2014, 53, 734; Zhou, J.; Hartwig, J. F. Angew. Chem. Int. Ed. 2008, 47, 578; Takahashi, M.; Oshima, K.; Matsubara, S. Chem. Lett. 2005, 34, 19; Neubert, L.; Michalik, D.; Bähn, S.; Imm, S.; Neumann, H.; Atzrodt, J.; Derdau, V.; Holla, W.; Beller, M. J. Am. Chem. Soc. 2012, 134, 12239; Sajiki, H.; I to, N.; Esaki, H.; Maesawa, T.; Maegawa, T.; Hirota, K. Tetrahedron Lett. 2005, 46, 6995; Sajiki, H.; Aoki, F.; Esaki, H.; Maegawa, T.; Hirota, K. Org. Lett. 2004, 6, 1485; US. Pat. Pub. No. 2004/0143126 (Webster et al.); Tsujino et al., Agric. Biol. Chem., 43 (4), 871-872, 1979; Huang et al., J Heterocyl. Chem. 2009 Nov. 6; 46(6): 1252-1258. The preceding articles are incorporated-by-reference into this document for all purposes.

Further references on synthetic methods for making deuterated-CBD compounds include: Ana Lago-Fernandez et al., New Methods for the Synthesis of Cannabidiol Derivatives, Methods in Enzymology, Volume 593, Chapter Eleven, pp 237-257. Kinney, W.A. et al. (2016). Discovery of KLS-13019, a cannabidiol-derived neuroprotective agent, with improved potency, safety, and permeability. ACS Medicinal Chemistry Letters, 7, 424-428. Klotter et al. (2015). Short and divergent total synthesis of (+)-machaerio B, (+)-machaeriol D, (+)-delta-8-THC, and analogues. Angewandte Chemie (International ed. In English), 54, 8547-8550. Kozela et al. (2016). HU-446 and HU-465, Derivatives of the nonpsychoactive cannabinoid cannabidiol, decrease the activation of encephalitogenic T Cells. Chemical Biology & Drug Design, 87, 143-153. Morales et al. (2017). An Overview on Medicinal Chemistry of Synthetic and Natural Derivatives of Cannabidiol. Frontiers in Pharmacology, June 2017, Volume 8, Article 422. Usami et al. (1999). Synthesis and pharmacological evaluation in mice of halogenated cannabidiol derivatives. Chem. Pharm. Bull. 47, 1641-1645, doi: 10.1248/cpb. 47.1641. Mechoulam et al. (2008). Novel Cannabidiol Derivatives and their use as anti-inflammatory agents. International Patent No WO2008/107879. Washington, D.C. Carlini et al. (1975). Anticonvulsant activity of four oxygenated cannabidiol derivatives. Res. Coomun. Chem. Pathol. Pharmacol. 12, 1-15. Mechoulam et al. (2002). Cannabidiol: an overview of some chemical and pharmacological aspects. Part I: chemical aspects. Chemistry and Physics of Lipids 121 (2002) 35-43. Lander et al. (1976). Total synthesis of cannabidiol and Δ1-THC Metabolites. J. Chem. Soc., Perkin I, 8-16. Harvey et al. (1990). Metabolites of cannabidiol identified in human urine. Xenobiotica 20, 303-320. Harvey et al. (1991). Urinary metabolites of cannabidiol in dog, rat and man and their identification by gas chromatography mass spectrometry. J. Chromatogr. Biomed. Appl. 562, 299-322. Mutsumi et al. (2011) Halogen-deuterium exchange reaction mediated by tributyltin hydride using THF-d8 as the deuterium source. Tetrahedron 67 (2011) 1158-1165. Dialer et al. (2017). Process for the Production of Cannabidiol and Delta-9-Tetrahydrocannabinol. US. Pat. Pub. No. 2017/0008868. The preceding articles are incorporated-by-reference into this document for all purposes.

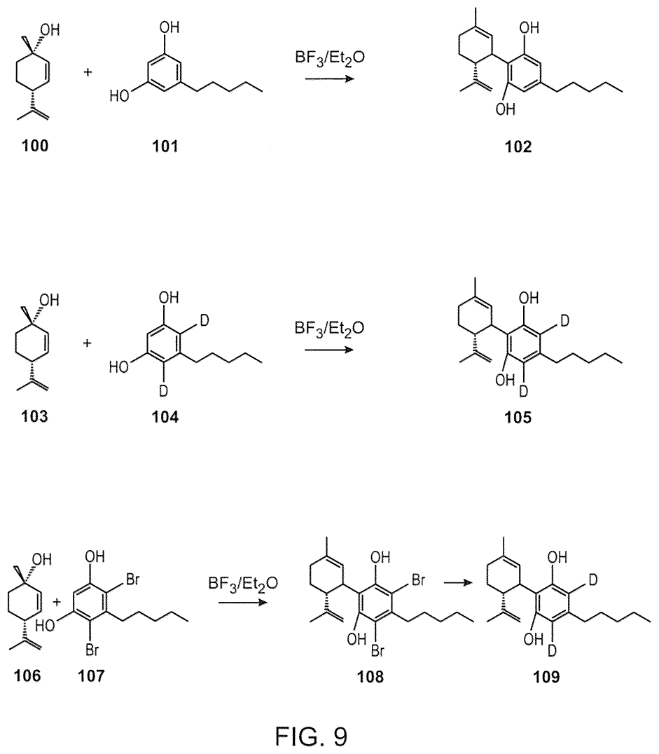

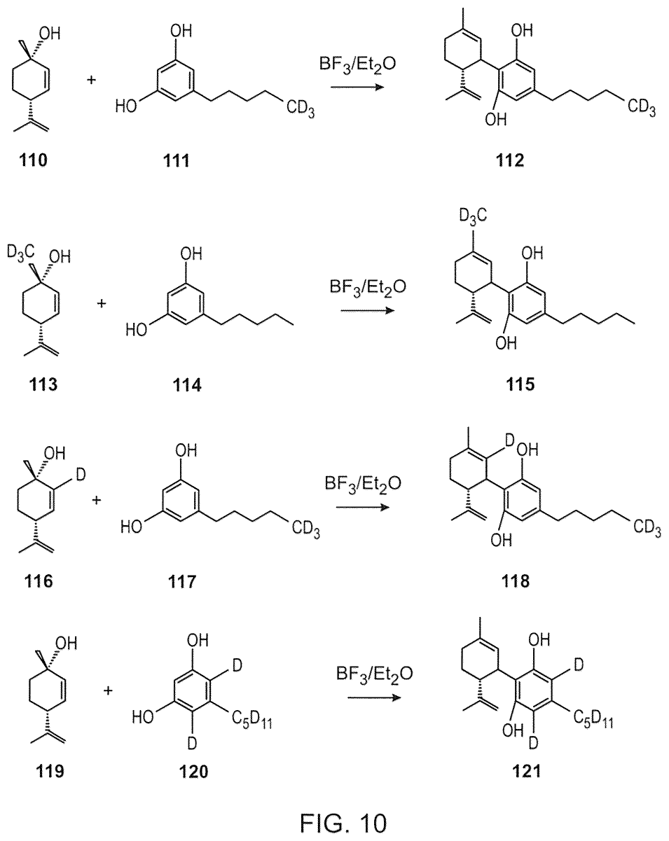

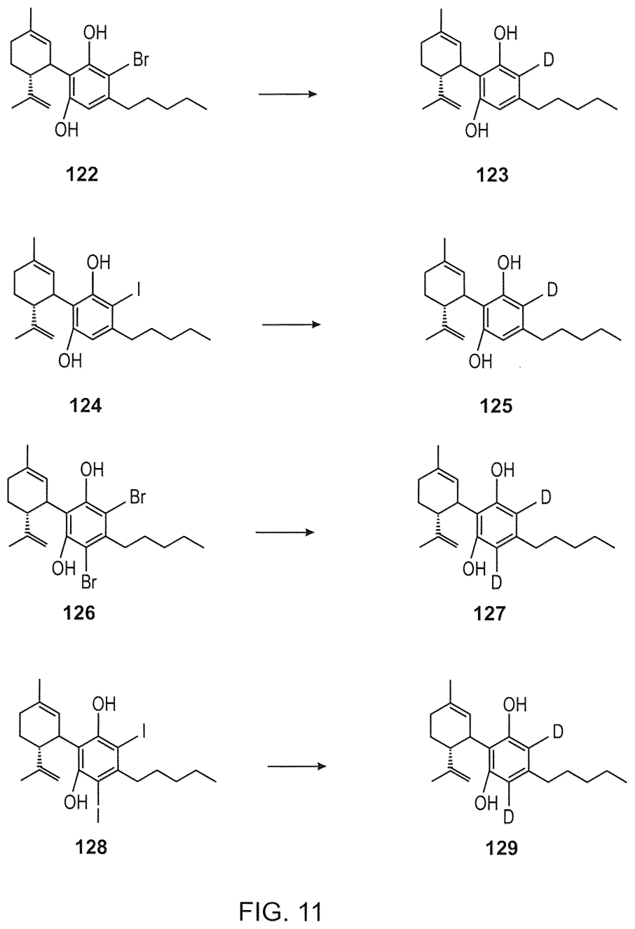

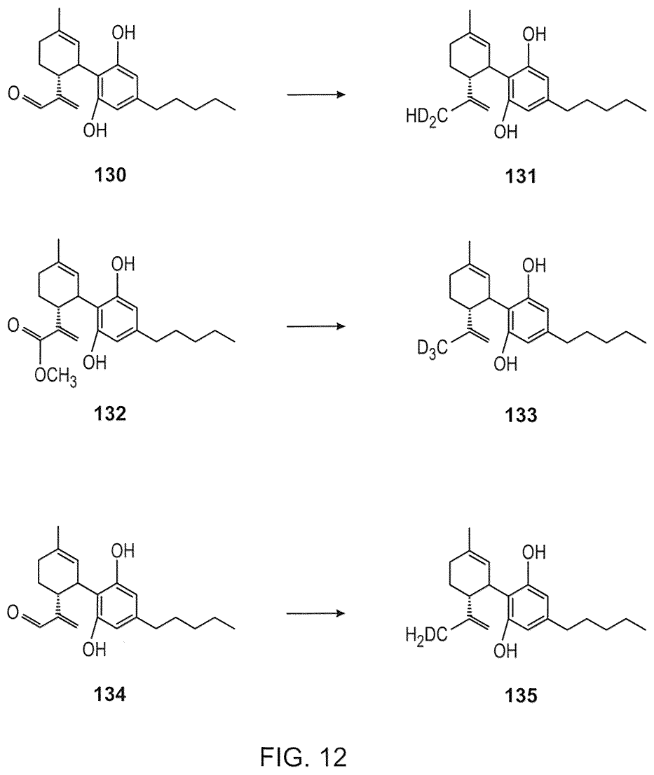

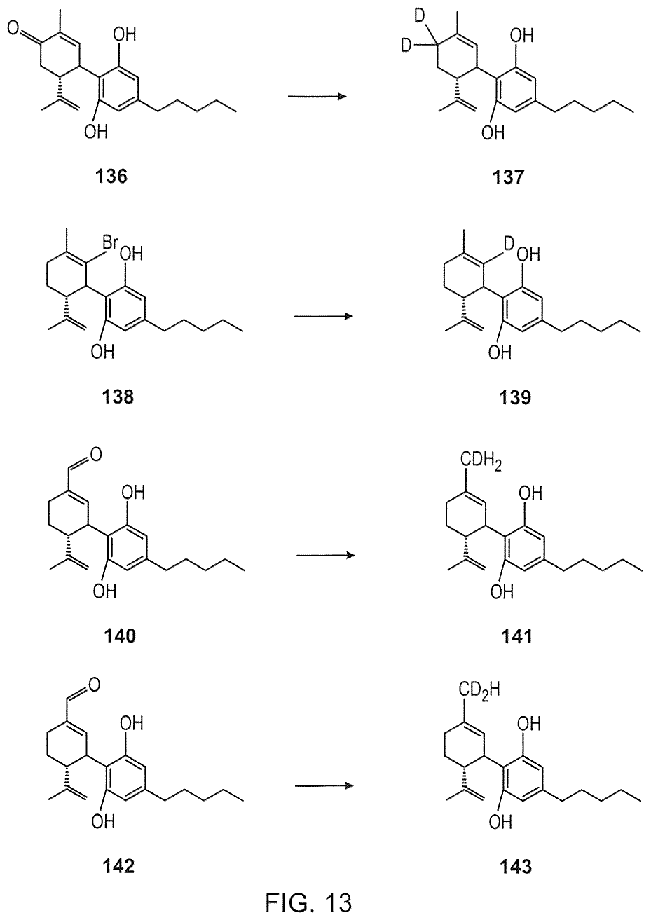

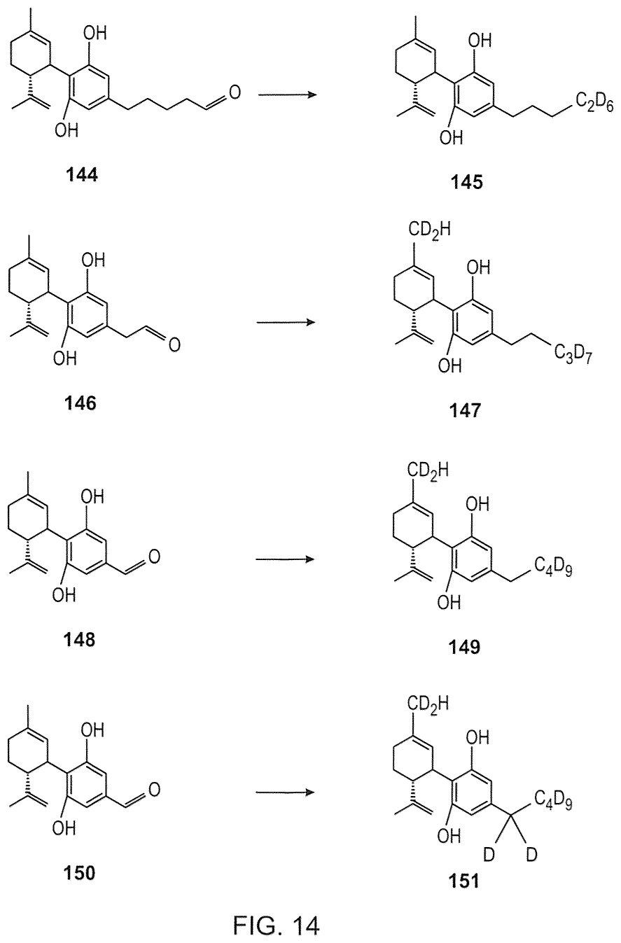

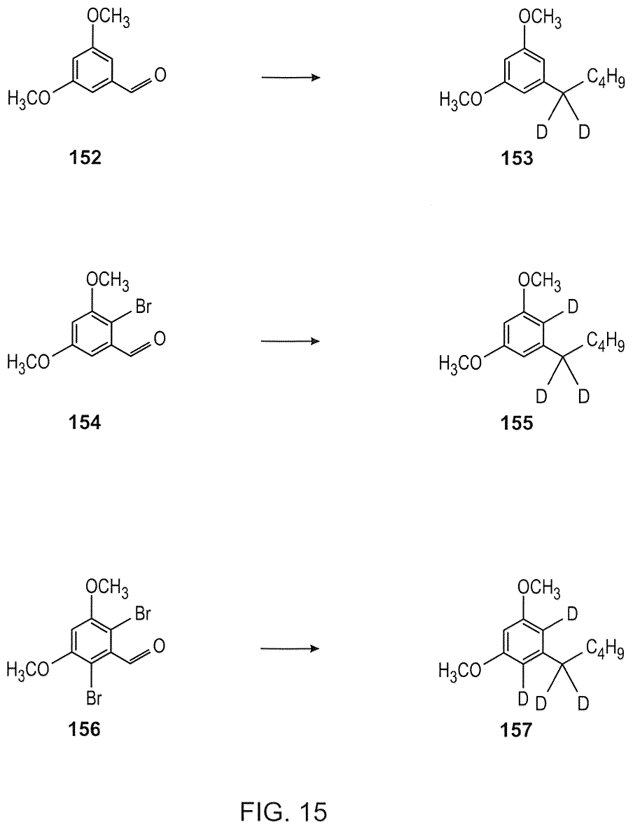

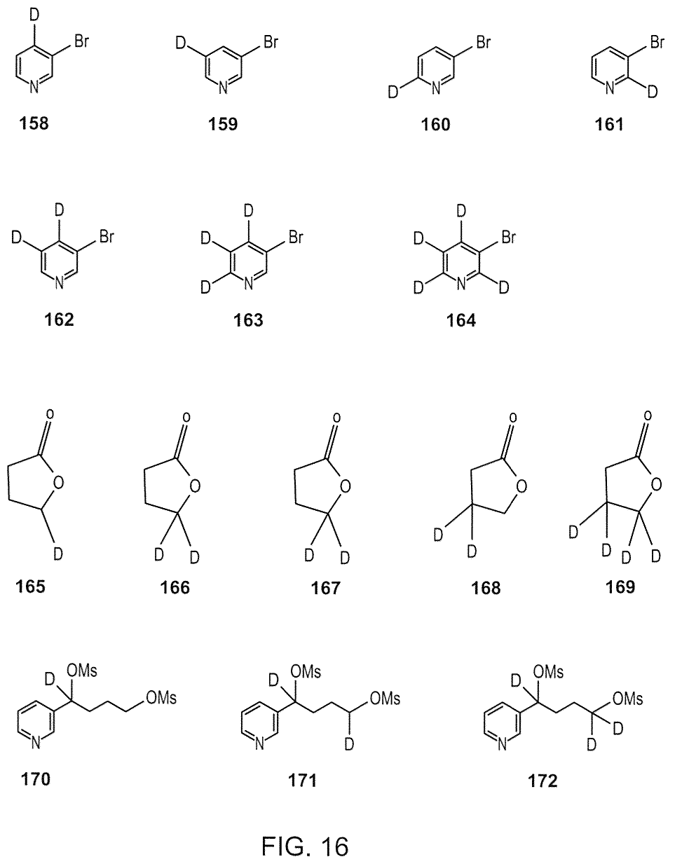

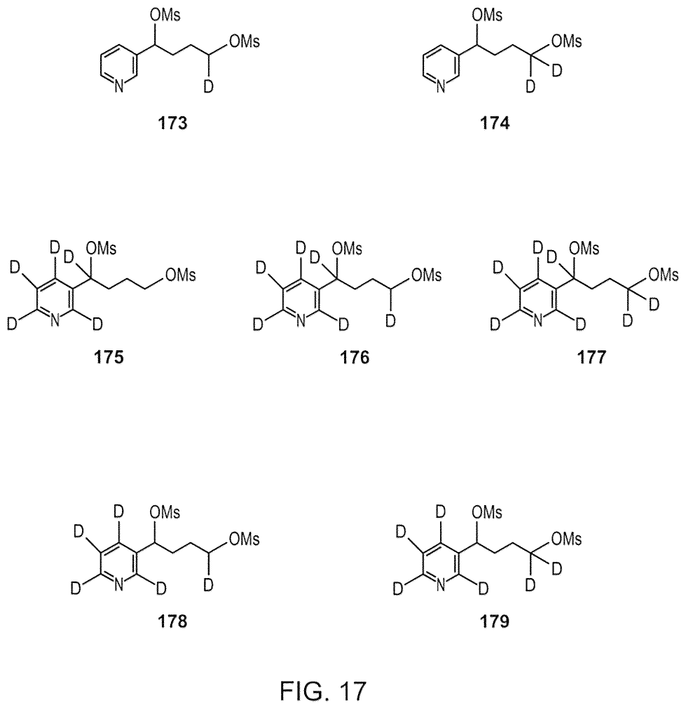

Examples of synthetic routes to deteurated cannabidiols are shown in FIGS. 9-14, with certain synthetic intermediates shown in FIG. 15. Deuterated tetrahydrocannabinols can be made, for example, from deuterated cannabidiols. See U.S. Pat. Pub. No. 2004/0143126 (Webster et al.). Examples of intermediates for the synthesis of deuterated nicotine are shown in FIGS. 16-17. For one synthetic route for nicotine, See Huang, et al., J. Heterocycl. Chem. 2009 Nov. 6; 46(6): 1252-1258. Deuterated cotinine can be made, for example, from deuterated nicotine. See Tsujino et al., Agric. Biol. Chem., 43(4), 871-872, 1979. The preceding articles are incorporated-by-reference into this document for all purposes.

The following are examples of combination compositions:
1. A composition comprising at least one of compound 3, 4, 5, 6, or 7. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
2. A composition comprising at least one of compound 8, 9, 10, 11, 12, 13, 14, 15 or 16. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
3. A composition comprising at least one of compound 17, 18, 19, 20, 21, 22, 23, 24 or 25. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
4. A composition comprising at least one of compound 26, 27, 28, 29, 30, 31, 32, 33 or 34. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
5. A composition comprising at least one of compound 37, 38, 39, 40, 41 or 42. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
6. A composition comprising at least one of compound 43, 44, 45, 46, 47, 48, 49 or 50. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
7. A composition comprising at least one of compound 53, 54, 55, 56, 57, 58, 59 or 60. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
8. A composition comprising at least one of compound 63, 64, 65, 66, 67, 68, 69 or 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
9. A composition comprising compound 3 and compound 37. A composition comprising compound 3 and compound 38. A composition comprising compound 3 and compound 39. A composition comprising compound 3 and compound 40. A composition comprising compound 3 and compound 41. A composition comprising compound 3 and compound 42. For the deuterated compounds (where “D” is indicated) the amount of deuterium present, as opposed to protium or tritium, is more than 70 percent. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
10. A composition comprising compound 4 and compound 37. A composition comprising compound 4 and compound 38. A composition comprising compound 4 and compound 39. A composition comprising compound 4 and compound 40. A composition comprising compound 4 and compound 41. A composition comprising compound 4 and compound 42. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
11. A composition comprising compound 5 and compound 37. A composition comprising compound 5 and compound 38. A composition comprising compound 5 and compound 39. A composition comprising compound 5 and compound 40. A composition comprising compound 5 and compound 41. A composition comprising compound 5 and compound 42. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
12. A composition comprising compound 6 and compound 37. A composition comprising compound 6 and compound 38. A composition comprising compound 6 and compound 39. A composition comprising compound 6 and compound 40. A composition comprising compound 6 and compound 41. A composition comprising compound 6 and compound 42. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
13. A composition comprising compound 7 and compound 37. A composition comprising compound 7 and compound 38. A composition comprising compound 7 and compound 39. A composition comprising compound 7 and compound 40. A composition comprising compound 7 and compound 41. A composition comprising compound 7 and compound 42. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
14. A composition comprising compound 53 and compound 63. A composition comprising compound 53 and compound 64. A composition comprising compound 53 and compound 65. A composition comprising compound 53 and compound 66. A composition comprising compound 53 and compound 67. A composition comprising compound 53 and compound 68. A composition comprising compound 53 and compound 69. A composition comprising compound 53 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
15. A composition comprising compound 54 and compound 63. A composition comprising compound 54 and compound 64. A composition comprising compound 54 and compound 65. A composition comprising compound 54 and compound 66. A composition comprising compound 54 and compound 67. A composition comprising compound 54 and compound 68. A composition comprising compound 54 and compound 69. A composition comprising compound 54 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
16. A composition comprising compound 55 and compound 63. A composition comprising compound 55 and compound 64. A composition comprising compound 55 and compound 65. A composition comprising compound 55 and compound 66. A composition comprising compound 55 and compound 67. A composition comprising compound 55 and compound 68. A composition comprising compound 55 and compound 69. A composition comprising compound 55 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
17. A composition comprising compound 56 and compound 63. A composition comprising compound 56 and compound 64. A composition comprising compound 56 and compound 65. A composition comprising compound 56 and compound 66. A composition comprising compound 56 and compound 67. A composition comprising compound 56 and compound 68. A composition comprising compound 56 and compound 69. A composition comprising compound 56 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
18. A composition comprising compound 57 and compound 63. A composition comprising compound 57 and compound 64. A composition comprising compound 57 and compound 65. A composition comprising compound 57 and compound 66. A composition comprising compound 57 and compound 67. A composition comprising compound 57 and compound 68. A composition comprising compound 57 and compound 69. A composition comprising compound 57 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
19. A composition comprising compound 58 and compound 63. A composition comprising compound 58 and compound 64. A composition comprising compound 58 and compound 65. A composition comprising compound 58 and compound 66. A composition comprising compound 58 and compound 67. A composition comprising compound 58 and compound 68. A composition comprising compound 58 and compound 69. A composition comprising compound 58 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
20. A composition comprising compound 59 and compound 63. A composition comprising compound 59 and compound 64. A composition comprising compound 59 and compound 65. A composition comprising compound 59 and compound 66. A composition comprising compound 59 and compound 67. A composition comprising compound 59 and compound 68. A composition comprising compound 59 and compound 69. A composition comprising compound 59 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
21. A composition comprising compound 60 and compound 63. A composition comprising compound 60 and compound 64. A composition comprising compound 60 and compound 65. A composition comprising compound 60 and compound 66. A composition comprising compound 60 and compound 67. A composition comprising compound 60 and compound 68. A composition comprising compound 60 and compound 69. A composition comprising compound 60 and compound 70. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
22. A composition comprising compound 3 and non-deuterium enriched tetrahydrocannabinol. A composition comprising compound 4 and non-deuterium enriched tetrahydrocannabinol. A composition comprising compound 5 and non-deuterium enriched tetrahydrocannabinol. A composition comprising compound 6 and non-deuterium enriched tetrahydrocannabinol. A composition comprising compound 7 and non-deuterium enriched tetrahydrocannabinol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
23. A composition comprising compound 8 and non-deuterium enriched tetrahydrocannabinol. A composition comprising compound 9 and non-deuterium enriched tetrahydrocannabinol. A composition comprising compound 10 and non-deuterium enriched tetrahydrocannabinol. A composition comprising compound 11 and non-deuterium enriched tetrahydrocannabinol. A composition comprising compound 12 and non-deuterium enriched tetrahydrocannabinol. A composition comprising compound 13 and non-deuterium enriched tetrahydrocannabinol. A composition comprising compound 14 and non-deuterium enriched tetrahydrocannabinol. A composition comprising compound 15 and non-deuterium enriched tetrahydrocannabinol. A composition comprising compound 16 and non-deuterium enriched tetrahydrocannabinol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
24. A composition comprising compound 17 and non-deuterium enriched tetrahydrocannabinol. A composition comprising compound 18 and non-deuterium enriched tetrahydrocannabinol. A composition comprising compound 19 and non-deuterium enriched tetrahydrocannabinol. A composition comprising compound 20 and non-deuterium enriched tetrahydrocannabinol. A composition comprising compound 21 and non-deuterium enriched tetrahydrocannabinol. A composition comprising compound 22 and non-deuterium enriched tetrahydrocannabinol. A composition comprising compound 23 and non-deuterium enriched tetrahydrocannabinol. A composition comprising compound 24 and non-deuterium enriched tetrahydrocannabinol. A composition comprising compound 25 and non-deuterium enriched tetrahydrocannabinol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
25. A composition comprising compound 26 and non-deuterium enriched tetrahydrocannabinol. A composition comprising compound 27 and non-deuterium enriched tetrahydrocannabinol. A composition comprising compound 28 and non-deuterium enriched tetrahydrocannabinol. A composition comprising compound 29 and non-deuterium enriched tetrahydrocannabinol. A composition comprising compound 30 and non-deuterium enriched tetrahydrocannabinol. A composition comprising compound 31 and non-deuterium enriched tetrahydrocannabinol. A composition comprising compound 32 and non-deuterium enriched tetrahydrocannabinol. A composition comprising compound 33 and non-deuterium enriched tetrahydrocannabinol. A composition comprising compound 34 and non-deuterium enriched tetrahydrocannabinol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
26. A composition comprising compound 37 and non-deuterium enriched cannabidiol. A composition comprising compound 38 and non-deuterium enriched cannabidiol. A composition comprising compound 39 and non-deuterium enriched cannabidiol. A composition comprising compound 40 and non-deuterium enriched cannabidiol. A composition comprising compound 41 and non-deuterium enriched cannabidiol. A composition comprising compound 42 and non-deuterium enriched cannabidiol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
27. A composition comprising compound 43 and non-deuterium enriched cannabidiol. A composition comprising compound 44 and non-deuterium enriched cannabidiol. A composition comprising compound 45 and non-deuterium enriched cannabidiol. A composition comprising compound 46 and non-deuterium enriched cannabidiol. A composition comprising compound 47 and non-deuterium enriched cannabidiol. A composition comprising compound 48 and non-deuterium enriched cannabidiol. A composition comprising compound 49 and non-deuterium enriched cannabidiol. A composition comprising compound 50 and non-deuterium enriched cannabidiol. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
28. A composition comprising compound 53 and non-deuterium enriched cotinine. A composition comprising compound 54 and non-deuterium enriched cotinine. A composition comprising compound 55 and non-deuterium enriched cotinine. A composition comprising compound 56 and non-deuterium enriched cotinine. A composition comprising compound 57 and non-deuterium enriched cotinine. A composition comprising compound 58 and non-deuterium enriched cotinine. A composition comprising compound 59 and non-deuterium enriched cotinine. A composition comprising compound 60 and non-deuterium enriched cotinine. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
29. A composition comprising compound 63 and non-deuterium enriched nicotine. A composition comprising compound 64 and non-deuterium enriched nicotine. A composition comprising compound 65 and non-deuterium enriched nicotine. A composition comprising compound 66 and non-deuterium enriched nicotine. A composition comprising compound 67 and non-deuterium enriched nicotine. A composition comprising compound 68 and non-deuterium enriched nicotine. A composition comprising compound 69 and non-deuterium enriched nicotine. A composition comprising compound 70 and non-deuterium enriched nicotine. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
30. A composition comprising compound 3 and non-deuterium enriched nicotine. A composition comprising compound 4 and non-deuterium enriched nicotine. A composition comprising compound 5 and non-deuterium enriched nicotine. A composition comprising compound 6 and non-deuterium enriched nicotine. A composition comprising compound 7 and non-deuterium enriched nicotine. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.
31. A composition comprising compound 8 and non-deuterium enriched nicotine. A composition comprising compound 9 and non-deuterium enriched nicotine. A composition comprising compound 10 and non-deuterium enriched nicotine. A composition comprising compound 11 and non-deuterium enriched nicotine. A composition comprising compound 12 and non-deuterium enriched nicotine. A composition comprising compound 13 and non-deuterium enriched nicotine. A composition comprising compound 14 and non-deuterium enriched nicotine. A composition comprising compound 15 and non-deuterium enriched nicotine. A composition comprising compound 16 and non-deuterium enriched nicotine. For the deuterated compounds (where “D” is indicated) the amount of deuterium present, as opposed to protium or tritium, is more than 70 percent. The composition optionally includes a colorant and/or flavorant. The composition comprises at least 1 mg of the at least one compound.

The following are examples of methods:
1. A method of treating anxiety, wherein the method comprises administration and/or ingestion and/or inhalation of one or more of compounds 1, 2, 3, 4, 5, 6 or 7 at a dose ranging from 1 mg/day to 275 mg/day to provide a beneficial change in symptoms as measured by the Beck Anxiety Inventory or another suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
2. A method of treating depression, wherein the method comprises administration and/or ingestion and/or inhalation of one or more of compounds 1, 2, 3, 4, 5, 6 or 7 at a dose ranging from 1 mg/day to 275 mg/day to provide a beneficial change in symptoms as measured by the Montgomery-Asberg Depression Rating Scale or another suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
3. A method of treating Sturge-Weber syndrome, wherein the method comprises administration and/or ingestion and/or inhalation of one or more compounds 1, 2, 3, 4, 5, 6 or 7 at a dose of 0.1 mg/kg/day to 20 mg/kg/day to observe a reduction in seizure frequency, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
4. A method of treating Treatment-Resistant Childhood Absence Seizures, wherein the method comprises administration and/or ingestion and/or inhalation of one or more compounds 1, 2, 3, 4, 5, 6 or 7 at a dose of 0.1 mg/kg/day to 35 mg/kg/day to observe a reduction in Absence Seizure Counts assessed by Video-electroencephalogram or other suitable measure, wherein the effect provided by the deutero compound is provided at a dose that is at least 10 percent less than the dose of the same non-deutero compound needed to produce the same effect.
5. A method of treating Graft Versus Host Disease, wherein the method comprises administration and/or ingestion and/or inhalation of one or more compounds 1, 2, 3, 4, 5, 6, or 7 at a dose ranging from 1.0 mg twice daily to 7.5 mg twice daily to observe a reduction in percentage of patients with acute GVHD, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
6. A method of treating Prader-Willi Syndrome, wherein the method comprises administration and/or ingestion and/or inhalation of one or more compounds 1, 2, 3, 4, 5, 6 or 7 at a dose ranging from 0.1 mg/kg/day to 15 mg/kg/day to observe a reduction in months to walking and/or months to talking, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
7. A method of treating Drug-Resistant Epilepsy, wherein the method comprises administration and/or ingestion and/or inhalation of one or more compounds 1, 2, 3, 4, 5, 6 or 7 at a dose of 0.1 mg/kg/day to 35 mg/kg/day to observe a reduction in seizures, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
8. A method of treating Inflammatory Bowel Disease (Chron's Disease or Ulcerative Colitis), wherein the method comprises administration and/or ingestion and/or inhalation of one or more compounds 1, 2, 3, 4, 5, 6 or 7 at a dose ranging from 1 mg twice daily to 4 mg twice daily to observe a reduction in Crohn's Disease Activity Index or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
9. A method of treating Refractory Infantile Spasms, wherein the method comprises administration and/or ingestion and/or inhalation of one or more compounds 1, 2, 3, 4, 5, 6 or 7 at a dose ranging from 0.1 mg/kg to 35 mg/kg twice a day to observe a reduction of spasms and/or hypsarrythmia as confirmed by video-electroencephalogram or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
10. A method of treating Dravet Syndrome, wherein the method comprises administration and/or ingestion and/or inhalation of one or more compounds 1, 2, 3, 4, 5, 6 or 7 at a dose ranging from 0.1 mg/kg/day to 7.5 mg/kg/day to observe a reduction in the frequency of tonic-colonic, clonic and focal seizures with motor components, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
11. A method of treating Lennox-Gastaut Syndrome, wherein the method comprises administration and/or ingestion and/or inhalation of one or more compounds 1, 2, 3, 4, 5, 6 or 7 at a dose ranging from 0.1 mg/kg/day to 7.5 mg/kg/day to observe a reduction in frequency of motor seizures involving the trunk or extremities, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
12. A method of treating Chronic Pain, wherein the method comprises administration and/or ingestion and/or inhalation of one or more compounds 1, 2, 3, 4, 5, 6 or 7 at a dose ranging from 1 mg twice a day to 150 mg twice a day to observe pain relief, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
13. A method of treating Schizophrenia, wherein the method comprises administration and/or ingestion and/or inhalation of one or more compounds 1, 2, 3, 4, 5, 6 or 7 at a dose ranging from 1 mg twice a day to 150 mg twice a day to observe a beneficial change in the Positive and Negative Syndrome Scale or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
14. A method of treating Atherosclerosis, wherein the method comprises administration and/or ingestion and/or inhalation of one or more compounds 1, 2, 3, 4, 5, 6 or 7 at a dose ranging from 1 mg twice a day 150 mg twice a day, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
15. A method of treating Multiple Sclerosis, wherein the method comprises administration and/or ingestion and/or inhalation of one or more compounds 1, 2, 3, 4, 5, 6 or 7 at a dose ranging from 1.0 mg to 100 mg daily to observe a reduction in VAS scores or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
16. A method of treating Osteoarthritis, wherein the method comprises administration and/or ingestion and/or inhalation of one or more compounds 1, 2, 3, 4, 5, 6 or 7 at a dose ranging from 1 mg twice a day to 150 mg twice a day to observe a reduction in transient joint inflammation and/or MIA-induced joint pain, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
17. A method of treating Dystonia, wherein the method comprises administration and/or ingestion and/or inhalation of one or more compounds 1, 2, 3, 4, 5, 6 or 7 at a dose ranging from 1 mg to 500 mg/day to observe a reduction in dystonic symptoms, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
18. A method of treating anxiety, wherein the method comprises administration and/or ingestion and/or inhalation of one or more of compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg/day to 275 mg/day to provide a beneficial change in symptoms as measured by the Beck Anxiety Inventory or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
19. A method of treating depression, wherein the method comprises administration and/or ingestion and/or inhalation of one or more of compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg/day to 275 mg/day to provide a beneficial change in symptoms as measured by the Montgomery-Asberg Depression Rating Scale or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
20. A method of treating Sturge-Weber syndrome, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose of 0.1 mg/kg/day to 20 mg/kg/day to observe a reduction in seizure frequency, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
21. A method of treating Treatment-Resistant Childhood Absence Seizures, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose of 0.1 mg/kg/day to 35 mg/kg/day to observe a reduction in Absence Seizure Counts assessed by Video-electroencephalogram or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
22. A method of treating Graft Versus Host Disease, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice daily to 7.5 mg twice daily to observe a reduction in percentage of patients with acute GVHD, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
23. A method of treating Prader-Willi Syndrome, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 0.1 mg/kg/day to 15 mg/kg/day to observe a reduction in months to walking and/or months to talking, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
24. A method of treating Drug-Resistant Epilepsy, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose of 0.1 mg/kg/day to 35 mg/kg/day to observe a reduction in seizures, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
25. A method of treating Inflammatory Bowel Disease (Chron's Disease or Ulcerative Colitis), wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice daily to 4 mg twice daily to observe a reduction in Crohn's Disease Activity Index or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
26. A method of treating Refractory Infantile Spasms, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 0.1 mg/kg to 35 mg/kg twice a day to observe a reduction of spasms and/or hypsarrythmia as confirmed by video-electroencephalogram or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
27. A method of treating Dravet Syndrome, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 0.1 mg/kg/day 7.5 mg/kg/day to observe a reduction in the frequency of tonic-colonic, clonic and focal seizures with motor components, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
28. A method of treating Lennox-Gastaut Syndrome, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 0.1 mg/kg/day to 7.5 mg/kg/day to observe a reduction in frequency of motor seizures involving the trunk or extremities, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
29. A method of treating Chronic Pain, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice a day to 150 mg twice a day to observe pain relief, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
30. A method of treating Schizophrenia, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice a day to 150 mg twice a day to observe a beneficial change in the Positive and Negative Syndrome Scale or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
31. A method of treating Atherosclerosis, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice a day to 150 mg twice a day, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
32. A method of treating Multiple Sclerosis, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1.0 mg to 100 mg daily to observe a reduction in VAS scores or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
33. A method of treating Osteoarthritis, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg twice a day to 150 mg twice a day to observe a reduction in transient joint inflammation and/or MIA-induced joint pain, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
34. A method of treating Dystonia, wherein the method comprises administration and/or ingestion and/or inhalation of one or more compounds 8, 9, 10, 11, 12, 13, 14, 15, 16, 17, 18, 19, 20, 21, 22, 23, 24, 25, 26, 27, 28, 29, 30, 31, 32, 33 or 34 at a dose ranging from 1 mg to 500 mg/day to observe a reduction in dystonic symptoms, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
35. A method of treating anxiety, wherein , wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
36. A method of treating glaucoma, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
37. A method of treating insomnia, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
38. A method of treating low appetite, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
39. A method of treating muscle spasticity, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
40. A method of treating nausea, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
41. A method of treating pain, wherein the method comprises administration and/or ingestion and/or inhalation of one or more compounds 35, 36, 37, 38, 39, 40, 41 or 42 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
42. A method of treating anxiety, wherein, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
43. A method of treating glaucoma, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
44. A method of treating insomnia, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
45. A method of treating low appetite, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
46. A method of treating muscle spasticity, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
47. A method of treating nausea, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
48. A method of treating pain, wherein the method comprises administration and/or ingestion and/or inhalation of one or more compounds 43, 44, 45, 46, 47, 48, 49 or 50 at a dose ranging between 1 mg and 100 mg, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
49. A method of treating anxiety, wherein the method comprises administration and/or ingestion and/or inhalation of a composition comprising compound 3 and compound 37, or compound 3 and compound 38, or compound 3 and compound 39, or compound 3 and compound 40, or compound 3 and compound 41, or compound 3 and compound 42, or compound 3 and compound 43, or compound 3 and compound 44, or compound 3 and compound 45, or compound 3 and compound 46, or compound 3 and compound 47, or compound 3 and compound 48, or compound 3 and compound 49, or compound 3 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
50. A method of treating anxiety, wherein the method comprises administration and/or ingestion and/or inhalation of a composition comprising compound 4 and compound 37, or compound 4 and compound 38, or compound 4 and compound 39, or compound 4 and compound 40, or compound 4 and compound 41, or compound 4 and compound 42, or compound 4 and compound 43, or compound 4 and compound 44, or compound 4 and compound 45, or compound 4 and compound 46, or compound 4 and compound 47, or compound 4 and compound 48, or compound 4 and compound 49, or compound 4 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
51. A method of treating anxiety, wherein the method comprises administration and/or ingestion and/or inhalation of a composition comprising compound 5 and compound 37, or compound 5 and compound 38, or compound 5 and compound 39, or compound 5 and compound 40, or compound 5 and compound 41, or compound 5 and compound 42, or compound 5 and compound 43, or compound 5 and compound 44, or compound 5 and compound 45, or compound 5 and compound 46, or compound 5 and compound 47, or compound 5 and compound 48, or compound 5 and compound 49, or compound 5 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
52. A method of treating anxiety, wherein the method comprises administration and/or ingestion and/or inhalation of a composition comprising compound 6 and compound 37, or compound 6 and compound 38, or compound 6 and compound 39, or compound 6 and compound 40, or compound 6 and compound 41, or compound 6 and compound 42, or compound 6 and compound 43, or compound 6 and compound 44, or compound 6 and compound 45, or compound 6 and compound 46, or compound 6 and compound 47, or compound 6 and compound 48, or compound 6 and compound 49, or compound 6 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
53. A method of treating anxiety, wherein the method comprises administration and/or ingestion and/or inhalation of a composition comprising compound 7 and compound 37, or compound 7 and compound 38, or compound 7 and compound 39, or compound 7 and compound 40, or compound 7 and compound 41, or compound 7 and compound 42, or compound 7 and compound 43, or compound 7 and compound 44, or compound 7 and compound 45, or compound 7 and compound 46, or compound 7 and compound 47, or compound 7 and compound 48, or compound 7 and compound 49, or compound 7 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
54. A method of treating anxiety, wherein the method comprises administration and/or ingestion and/or inhalation of a composition comprising compound 9 and compound 37, or compound 9 and compound 38, or compound 9 and compound 39, or compound 9 and compound 40, or compound 9 and compound 41, or compound 9 and compound 42, or compound 9 and compound 43, or compound 9 and compound 44, or compound 9 and compound 45, or compound 9 and compound 46, or compound 9 and compound 47, or compound 9 and compound 48, or compound 9 and compound 49, or compound 9 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
55. A method of treating anxiety, wherein the method comprises administration and/or ingestion and/or inhalation of a composition comprising compound 15 and compound 37, or compound 15 and compound 38, or compound 15 and compound 39, or compound 15 and compound 40, or compound 15 and compound 41, or compound 15 and compound 42, or compound 15 and compound 43, or compound 15 and compound 44, or compound 15 and compound 45, or compound 15 and compound 46, or compound 15 and compound 47, or compound 15 and compound 48, or compound 15 and compound 49, or compound 15 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
56. A method of treating anxiety, wherein the method comprises administration and/or ingestion and/or inhalation of a composition comprising compound 17 and compound 37, or compound 17 and compound 38, or compound 17 and compound 39, or compound 17 and compound 40, or compound 17 and compound 41, or compound 17 and compound 42, or compound 17 and compound 43, or compound 17 and compound 44, or compound 17 and compound 45, or compound 17 and compound 46, or compound 17 and compound 47, or compound 17 and compound 48, or compound 17 and compound 49, or compound 17 and compound 50 at a combined dose ranging from 1 mg to 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
57. A method of treating depression, wherein the method comprises administration and/or ingestion and/or inhalation of a composition comprising compound 3 and compound 37, or compound 3 and compound 38, or compound 3 and compound 39, or compound 3 and compound 40, or compound 3 and compound 41, or compound 3 and compound 42, or compound 3 and compound 43, or compound 3 and compound 44, or compound 3 and compound 45, or compound 3 and compound 46, or compound 3 and compound 47, or compound 3 and compound 48, or compound 3 and compound 49, or compound 3 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
58. A method of treating depression, wherein the method comprises administration and/or ingestion and/or inhalation of a composition comprising compound 4 and compound 37, or compound 4 and compound 38, or compound 4 and compound 39, or compound 4 and compound 40, or compound 4 and compound 41, or compound 4 and compound 42, or compound 4 and compound 43, or compound 4 and compound 44, or compound 4 and compound 45, or compound 4 and compound 46, or compound 4 and compound 47, or compound 4 and compound 48, or compound 4 and compound 49, or compound 4 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
59. A method of treating depression, wherein the method comprises administration and/or ingestion and/or inhalation of a composition comprising compound 5 and compound 37, or compound 5 and compound 38, or compound 5.and compound 39, or compound 5 and compound 40, or compound 5 and compound 41, or compound 5 and compound 42, or compound 5 and compound 43, or compound 5 and compound 44, or compound 5 and compound 45, or compound 5 and compound 46, or compound 5 and compound 47, or compound 5 and compound 48, or compound 5 and compound 49, or compound 5 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
60. A method of treating depression, wherein the method comprises administration and/or ingestion and/or inhalation of a composition comprising compound 6 and compound 37, or compound 6 and compound 38, or compound 6 and compound 39, or compound 6 and compound 40, or compound 6 and compound 41, or compound 6 and compound 42, or compound 6 and compound 43, or compound 6 and compound 44, or compound 6 and compound 45, or compound 6 and compound 46, or compound 6 and compound 47, or compound 6 and compound 48, or compound 6 and compound 49, or compound 6 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
61. A method of treating depression, wherein the method comprises administration and/or ingestion and/or inhalation of a composition comprising compound 7 and compound 37, or compound 7 and compound 38, or compound 7 and compound 39, or compound 7 and compound 40, or compound 7 and compound 41, or compound 7 and compound 42, or compound 7 and compound 43, or compound 7 and compound 44, or compound 7 and compound 45, or compound 7 and compound 46, or compound 7 and compound 47, or compound 7 and compound 48, or compound 7 and compound 49, or compound 7 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
62. A method of treating depression, wherein the method comprises administration and/or ingestion and/or inhalation of a composition comprising compound 9 and compound 37, or compound 9 and compound 38, or compound 9 and compound 39, or compound 9 and compound 40, or compound 9 and compound 41, or compound 9 and compound 42, or compound 9 and compound 43, or compound 9 and compound 44, or compound 9 and compound 45, or compound 9 and compound 46, or compound 9 and compound 47, or compound 9 and compound 48, or compound 9 and compound 49, or compound 9 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
63. A method of treating depression, wherein the method comprises administration and/or ingestion and/or inhalation of a composition comprising compound 15 and compound 37, or compound 15 and compound 38, or compound 15 and compound 39, or 1 compound 15 and compound 40, or compound 15 and compound 41, or compound 15 and compound 42, or compound 15 and compound 43, or compound 15 and compound 44, or compound 15 and compound 45, or compound 15 and compound 46, or compound 15 and compound 47, or compound 15 and compound 48, or compound 15 and compound 49, or compound 15 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
64. A method of treating depression, wherein the method comprises administration and/or ingestion and/or inhalation of a composition comprising compound 17 and compound 37, or compound 17 and compound 38, or compound 17 and compound 39, or compound 17 and compound 40, or compound 17 and compound 41, or compound 17 and compound 42, or compound 17 and compound 43, or compound 17 and compound 44, or compound 17 and compound 45, or compound 17 and compound 46, or compound 17 and compound 47, or compound 17 and compound 48, or compound 17 and compound 49, or compound 17 and compound 50 at a combined dose ranging from 1 mg to 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
65. A method of treating Treatment-Resistant Childhood Absence Seizures, wherein the method comprises administration and/or ingestion and/or inhalation of a composition comprising compound 3 and compound 37, or compound 3 and compound 38, or compound 3 and compound 39, or compound 3 and compound 40, or compound 3 and compound 41, or compound 3 and compound 42, or compound 3 and compound 43, or compound 3 and compound 44, or compound 3 and compound 45, or compound 3 and compound 46, or compound 3 and compound 47, or compound 3 and compound 48, or compound 3 and compound 49, or compound 3 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
66. A method of treating Treatment-Resistant Childhood Absence Seizures, wherein the method comprises administration and/or ingestion and/or inhalation of a composition comprising compound 4 and compound 37, or compound 4 and compound 38, or compound 4 and compound 39, or compound 4 and compound 40, or compound 4 and compound 41, or compound 4 and compound 42, or compound 4 and compound 43, or compound 4 and compound 44, or compound 4 and compound 45, or compound 4 and compound 46, or compound 4 and compound 47, or compound 4 and compound 48, or compound 4 and compound 49, or compound 4 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
67. A method of treating Treatment-Resistant Childhood Absence Seizures, wherein the method comprises administration and/or ingestion and/or inhalation of a composition comprising compound 5 and compound 37, or compound 5 and compound 38, or compound 5 and compound 39, or compound 5 and compound 40, or compound 5 and compound 41, or compound 5 and compound 42, or compound 5 and compound 43, or compound 5 and compound 44, or compound 5 and compound 45, or compound 5 and compound 46, or compound 5 and compound 47, or compound 5 and compound 48, or compound 5 and compound 49, or compound 5 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
68. A method of treating Treatment-Resistant Childhood Absence Seizures, wherein the method comprises administration and/or ingestion and/or inhalation of a composition comprising compound 6 and compound 37, or compound 6 and compound 38, or compound 6 and compound 39, or compound 6 and compound 40, or compound 6 and compound 41, or compound 6 and compound 42, or compound 6 and compound 43, or compound 6 and compound 44, or compound 6 and compound 45, or compound 6 and compound 46, or compound 6 and compound 47, or compound 6 and compound 48, or compound 6 and compound 49, or compound 6 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
69. A method of treating Treatment-Resistant Childhood Absence Seizures, wherein the method comprises administration and/or ingestion and/or inhalation of a composition comprising compound 7 and compound 37, or compound 7 and compound 38, or compound 7 and compound 39, or compound 7 and compound 40, or compound 7 and compound 41, or compound 7 and compound 42, or compound 7 and compound 43, or compound 7 and compound 44, or compound 7 and compound 45, or compound 7 and compound 46, or compound 7 and compound 47, or compound 7 and compound 48, or compound 7 and compound 49, or compound 7 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
70. A method of treating Treatment-Resistant Childhood Absence Seizures, wherein the method comprises administration and/or ingestion and/or inhalation of a composition comprising compound 9 and compound 37, or compound 9 and compound 38, or compound 9 and compound 39, or compound 9 and compound 40, or compound 9 and compound 41, or compound 9 and compound 42, or compound 9 and compound 43, or compound 9 and compound 44, or compound 9 and compound 45, or compound 9 and compound 46, or compound 9 and compound 47, or compound 9 and compound 48, or compound 9 and compound 49, or compound 9 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
71. A method of treating Treatment-Resistant Childhood Absence Seizures, wherein the method comprises administration and/or ingestion and/or inhalation of a composition comprising compound 15 and compound 37, or compound 15 and compound 38, or compound 15 and compound 39, or compound 15 and compound 40, or compound 15 and compound 41, or compound 15 and compound 42, or compound 15 and compound 43, or compound 15 and compound 44, or compound 15 and compound 45, or compound 15 and compound 46, or compound 15 and compound 47, or compound 15 and compound 48, or compound 15 and compound 49, or compound 15 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
72. A method of treating Treatment-Resistant Childhood Absence Seizures, wherein the method comprises administration and/or ingestion and/or inhalation of a composition comprising compound 17 and compound 37, or compound 17 and compound 38, or compound 17 and compound 39, or compound 17 and compound 40, or compound 17 and compound 41, or compound 17 and compound 42, or compound 17 and compound 43, or compound 17 and compound 44, or compound 17 and compound 45, or compound 17 and compound 46, or compound 17 and compound 47, or compound 17 and compound 48, or compound 17 and compound 49, or compound 17 and compound 50 at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
73. A method of treating anxiety, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose between 1 mg/day and 275 mg/day to provide a beneficial change in symptoms as measured by the Beck Anxiety Inventory or other suitable measure. For the deuterated compounds (where “D” is indicated) the amount of deuterium present, as opposed to protium or tritium, is more than 70 percent.
74. A method of treating depression, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose between 1 mg/day and 275 mg/day to provide a beneficial change in symptoms as measured by the Montgomery-Asberg Depression Rating Scale or other suitable measure. For the deuterated compounds (where “D” is indicated) the amount of deuterium present, as opposed to protium or tritium, is more than 70 percent.
75. A method of treating Sturge-Weber syndrome, wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 1 mg/day to 20 mg/day to observe a reduction in seizure frequency. For the deuterated compounds (where “D” is indicated) the amount of deuterium present, as opposed to protium or tritium, is more than 70 percent.
76. A method of treating Treatment-Resistant Childhood Absence Seizures, wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 0.1 mg/kg/day to 35 mg/kg/day to observe a reduction in Absence Seizure Counts assessed by Video-electroencephalogram or other suitable measure. For the deuterated compounds (where “D” is indicated) the amount of deuterium present, as opposed to protium or tritium, is more than 70 percent.
77. A method of treating Graft Versus Host Disease, wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 1.0 mg to 7.5 mg twice daily to observe a reduction in percentage of patients with acute GVHD. For the deuterated compounds (where “D” is indicated) the amount of deuterium present, as opposed to protium or tritium, is more than 70 percent.
78. A method of treating Prader-Willi Syndrome, wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 0.1 mg/kg/day to 15 mg/kg/day to observe a reduction in months to walking or months to talking.
79. A method of treating Drug-Resistant Epilepsy, wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 0.1 mg/kg/day to 35 mg/kg/day to observe a reduction in seizures. For the deuterated compounds (where “D” is indicated) the amount of deuterium present, as opposed to protium or tritium, is more than 70 percent.
80. A method of treating Inflammatory Bowel Disease (Chron's Disease or Ulcerative Colitis), wherein the method comprises administration and/or ingestion and/or inhalation of a composition comprising one or more compounds 1, 2, 3, 4, 5, 6, 9, 15 or 17 and non-deuterium enriched tetrahydro-cannabinol at a combined dose ranging from 1 mg to 4 mg twice daily to observe a reduction in Chron's Disease Activity Index or other suitable measure. For the deuterated compounds (where “D” is indicated) the amount of deuterium present, as opposed to protium or tritium, is more than 70 percent.
81. A method of treating Refractory Infantile Spasms, wherein the method comprises wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 0.1 mg/kg to 35 mg/kg twice a day to observe a reduction of spasms and/or hypsarrythmia as confirmed by video-electroencephalogram or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
82. A method of treating Dravet Syndrome, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 0.1 mg/kg/day to 7.5 mg/kg/day to observe a reduction in the frequency of tonic-colonic, clonic and focal seizures with motor components, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
83. A method of treating Lennox-Gastaut Syndrome, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1.0 mg/kg/day to 7.5 mg/kg/day to observe a reduction in frequency of motor seizures involving the trunk or extremities, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
84. A method of treating Chronic Pain, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1 mg twice a day to 150 mg twice a day to observe pain relief, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
85. A method of treating Schizophrenia, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1 mg twice a day to 150 mg twice a day to observe a beneficial change in the Positive and Negative Syndrome Scale or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
86. A method of treating Atherosclerosis, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1 mg twice a day to 150 mg twice a day, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
87. A method of treating Multiple Sclerosis, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1.0 mg to 100 mg daily to observe a reduction in VAS scores or other suitable measure, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
88. A method of treating Osteoarthritis, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 1 mg twice a day to 150 mg twice a day to observe a reduction in transient joint inflammation and/or MIA-induced joint pain, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.
89. A method of treating Dystonia, wherein the method comprises administration and/or ingestion and/or inhalation of a composition comprising one or more compounds 1, 2, 3, 4, 5, 6, 9, 15 or 17 and non-deuterium enriched tetrahydrocannabinol at a combined dose ranging from 50 mg to 500 mg/day to observe a reduction in dystonic symptoms, wherein the effect provided by the deutero compound is provided at a dose that is at least 10 percent less than the dose of the same non-deutero compound needed to produce the same effect. For the deuterated compounds (where “D” is indicated) the amount of deuterium present, as opposed to protium or tritium, is more than 70 percent.

In certain embodiments, an appropriate dosage level for the compounds of the present invention is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

In certain cases, compounds of the present invention can be inhaled, typically as a vapor or aerosol. The vapor or aerosol can be produced by any suitable method, including use of an e-cigarette. Oftentimes when an e-cigarette is used, one or more compounds of the present invention are included in an e-liquid. Such an e-liquid typically contains at least: one or more compounds of the present invention; propylene glycol; glycerin; and one or more flavorings.

For oral administration of compounds and compositions of the present invention, any suitable form (e.g., tablet, oil, suspension, etc.) may be used.

### REFERENCES

Pohanka, Int. J. Mol. Sci., “Alpha7 Nicotinic Acetylcholine Receptor is a Target in Pharmacology and Toxicology”, 2012, 13(2), 2219-2238.

Abburi et al. FASEB, “Analgesic Effects Mediated by Alpha 7 Nicotinic Acetylcholine Receptors: A Potential Non-Opioid Treatment for Pain”, published online 1 Apr. 2017, abstract number Ib578.

Ren et al. Int. J. Biol. Sci., “The Protective Effect of Alpha 7 Nicotinic Acetylcholine Receptor Activation on Critical Illness and Its Mechanism”, 2017, 13(1), 46-56.

Liu et al. Cell Physiol Biochem, “Activation of α7 Nicotinic Acetylcholine Receptors Prevents Monosodium Iodoacetate-Induced Osteoarthritis in Rats”, 2015, 35, 627-638.

Treinen et al. Cent Nerv Syst Agents Med Chem, “Role of the α7 Nicotinic Acetylcholine Receptor and RIC-3 in the Cholinergic Anti-Inflammatory Pathway”, 2017, 17(2), 90-99.

Mandl et al. Brain Research Bulletin, “Role of presynaptic nicotinic acetylcholine receptors in the regulation of gastrointestinal motility”, 2007, 72(4-6), 194-200.

Kucinski et al. Schizophr Res. 2012 April;136(1-3):82-7.

Walling et al. Schizophr Bull. 2016 March;42(2):335-43.

The preceding articles and patent documents are incorporated-by-reference into this document for all purposes.

1. A compound, where the compound is of the structure below: 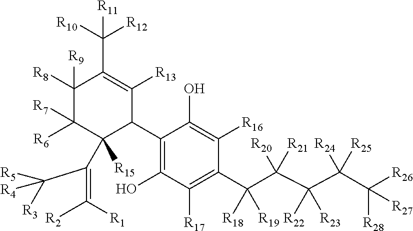
wherein, R₁ to R₂₈ are independently “H” or “D”, where at least one of the substituents is “D”.

2-15. (canceled)

16. A compound, where the compound is of the structure below: 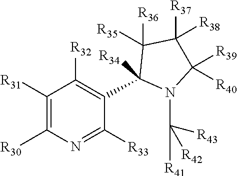
wherein, R₃₀ to R₄₃ are independently “H” or “D”, where at least one of the substituents is “D”.

17. The compound according to claim 16, wherein the compound is:

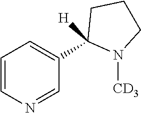 [SMILES: [2H]C([2H])([2H])N1CCC[C@H]1c1cccnc1]

18. The compound according to claim 16, wherein the compound is:

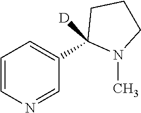

19. The compound according to claim 16, wherein the compound is:

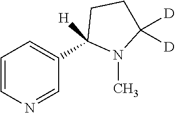

20. The compound according to claim 16, wherein the compound is:

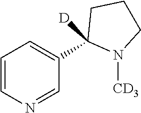 [SMILES: [2H]C([2H])([2H])N1CCC[C@@]1([2H])c1cccnc1]

21. The compound according to claim 16, wherein the compound is: 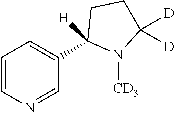 [SMILES: [2H]C([2H])([2H])N1[C@H](c2cccnc2)CCC1([2H])[2H]]
